SEQUENCE LISTING (3 sequences)

SEQ ID NO: 1 - DNA, 19 bp, Artificial Sequence
  oligonucleotide
acctgccctcctcattgca

SEQ ID NO: 2 - DNA, 17 bp, Artificial Sequence
  oligonucleotide
agtggactcgcgcatag

SEQ ID NO: 3 - protein, 21 aa, Artificial Sequence
  Amino acid sequence of Tertiapin (small protein derived from honeybee venom)
ALCNCNRIIIPHMCWKKCGKK

(19) United States
(12) Patent Application Publication
(10) Pub. No.: US 2007/0270352 A1
(43) Pub. Date: Nov. 22, 2007

(54) Method of Treatment

(76) Inventors: Perry Francis Bartlett, Albert Park (AU); Elizabeth Jane Coulson, St. Lucia (AU); Samuel Nicholas Morley, Highgate Hill (AU); Sarah Marie Hulett, Brighton (AU)

Correspondence Address: SEED INTELLECTUAL PROPERTY LAW GROUP PLLC, 701 FIFTH AVE, SUITE 5400, SEATTLE, WA, 98104, US

(21) Appl. No.: 10/514,374

(22) PCT Filed: May 14, 2003
(86) PCT No.: PCT/AU03/00580

§ 371 Date: Feb. 3, 2006

(30) Foreign Application Priority Data
May 14, 2002 (AU) PS 2307

(51) Int. Cl. A61K 31/44; A61K 31/7088; A61K 38/17; A61P 25/28

(52) U.S. Cl. 514/21; 514/330; 514/44

(57) ABSTRACT

The present invention relates generally to a method for preventing, inhibiting or otherwise reducing cell death. More particularly, the present invention contemplates a method for preventing, inhibiting or otherwise reducing neuronal cell death such as during neurodegenerative disease or following trauma The method of the present invention is generally practiced by the administration to a mammalian including a human subject, of an effective amount of an agent which blocks, retards or otherwise impairs ions from entering or passing through an ion channel. In one particular embodiment, the ion channel is a potassium (K) ion (K+) channel. The present invention further provides compositions comprising ion channel blockers and in particular K+ channel blockers. The compositions may also comprise other therapeutic agents such as agents which reduce levels of, or the activity of, a neurotrophin receptor. The present invention further provides methods for promoting cell survival by promoting intracellular cleavage of the neutrophil receptor by generating or introducing intracellular forms of the receptor such as by genetic or protein supplementation means. The present invention further provides a method for determining the likelihood of neurological cell degeneration by determining the level of function of K+ channels wherein an impaired ion channel is indicative of a reduced likelihood of neuronal cell apoptosis. Furthermore, the present invention provides antagonists of K+ channels, such as antagonists of molecules which mediate K+ channel activation via neurotrophin receptors or domains thereof.

### BACKGROUND OF THE INVENTION

1. Field of the Invention

The present invention relates generally to a method for preventing, inhibiting or otherwise reducing cell death. More particularly, the present invention contemplates a method for preventing, inhibiting or otherwise reducing neuronal cell death such as during neurodegenerative disease or following trauma. The method of the present invention is generally practiced by the administration, to a mammalian including a human subject, of an effective amount of an agent which blocks, retards or otherwise impairs ions from entering or passing through an ion channel. In one particular embodiment, the ion channel is a potassium (K) ion (K⁺) channel. The present invention further provides compositions comprising ion channel blockers and in particular K⁺ channel blockers. The compositions may also comprise other therapeutic agents such as agents which reduce levels of, or the activity of, a neurotrophin receptor. The present invention further provides methods for promoting cell survival by promoting intracellular cleavage of the neutrophil receptor by generating or introducing intracellular forms of the receptor such as by genetic or protein supplementation means. The present invention further provides a method for determining the likelihood of neurological cell degeneration by determining the level of function of K⁺ channels wherein an impaired ion channel is indicative of a reduced likelihood of neuronal cell apoptosis. Furthermore, the present invention provides antagonists of K⁺ channels, such as antagonists of molecules which mediate K⁺ channel activation via neurotrophin receptors or domains thereof.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Programmed cell death of neurons is well known to be involved in the correct formation of the nervous system. Programmed cell death is activated in neuronal populations around the times of synaptogenisis and the reliance of the neuron on various target derived growth factors such as the neurotrophin family (e.g. NGF, BDNF) or the neural cytokines (e.g. LIF, CNTF). It has been proposed that those neurons competing unsuccessfully for target derived growth factors or making incorrect connections will undergo programmed cell suicide. However, death does not occur until synaptic connections have been made. Consequently, blockade of activity can rescue neurons that would otherwise not get growth factors (Banks et al., J. Comp. Neurol. 429: 156-165, 2001; Terrado et al., J. Neurosci. 21: 3144-3510, 2001). Furthermore, neurons that have been removed from their trophic support can be kept alive either in depolarizing conditions (Yu and Choi, Proc. Natl. Acad. Sci. USA 97: 9360-9362, 2000) or by mimicking electrical activity by stimulating receptor pathways down-stream of neurotransmitters (Pereira et al., Int. J. Dv. Neurosci. 19: 559-567,2001).

Other facts which are involved in programmed cell death include electrical state of a neuron and ionic fluxing. In particular, efflux of some ions has been shown to be an integral part of both neuronal and non-neuronal cell death pathways.

One such ion studied is potassium. This ion appears to contribute to a range of 25 physiological phenomena including being a prerequisite to cell volume loss (Bortner et al., J. Biol. Chem. 276: 4304-4314, 2001), being coincident with the appearance of annexin V on the extracellular lipid membrane (D)allaporta et al., J. Immunol 160: 5605-5615, 1998); promoting activation of endonucleases leading to DNA laddering and cleavage of pro-caspase 3 to its active form (Hughes et al., J. Biol. Chem. 272: 30567-30576, 1997); and associated with activation of jun kinase phosphorylation pathways (Wang et al., J. Biol. Chem. 274: 3678-3685, 1999).

One of the proteins shown to be involved in mediating both naturally occurring and neurodegenerative cell death is the neurotrophin receptor, p75^{NTR}. In both isolated neurons and in vivo, increased and decreased levels of p75^{NTR} expression have been correlated with increased and decreased cell death, respectively. Activation of p75^{NTR} by neurotrophins (when the appropriate survival signaling trk receptor is not expressed) has also been shown in a wide range of neuronal types and glial. Currently, an expanding number of proteins are being identified as p75^{NTR}-associating proteins including Trafs, NRIF, SC-1, NADE, NRAGE and FAP-1. These proteins can promote both cell death (Trafs, NRWF, NADE, NRAGE) and mediate other cellular processes such as activation of NfkappaB (Trafs, sc-1). Interestingly, many of the identified proteins interact with the juxtaposed membrane region of p75^{NTR}, the “Chopper domain” region. This domain has been identified as a region which can have mediated cell death (Coulson et al., J. Biol. Chem. 275: 30537, 30545, 2000), as well as, or rather than, the region with homology to the Fas and TNFR death domain. However, the precise pathway by which any binding protein together with p75^{NTR} signals cell death remains unclear.

In work leading up to the present invention, the inventors investigated the role of ions and ion channels on cell death induced by the membrane-associated form of the Chopper domain of p75^{NTR}. In accordance with the present invention, it has been found that neurons require an efflux of ions for mediation of Chopper-induced cell death.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention provides a means for reducing cell degeneration and in particular neuronal cell degeneration. In accordance with the present invention, it has been determined that neuronal cell apoptosis or other forms of degeneration require the efflux of particular ions such as K⁺. The blocking of ion channels such as K⁺ channels reduces the incidence of neuronal cell degeneration. Without limiting the present invention to any one theory or mode of action, it is proposed that at least K⁺ is required for neuronal cell apoptosis mediated by p75^{NTR} or its Chopper domain. Blocking or reducing the effectiveness of K⁺ channels reduces p75^{NTR}-mediated and in particular Chopper domain-mediated apoptosis.

Consequently, in one embodiment, the present invention contemplates a method for reducing cell death and in particular neuronal cell death in a subject by the administration of an ion channel blocking agent such as a K⁺ channel blocker. Reference to a “blocker” or a “blocking agent” is not intended to imply that there is complete inhibition of the functioning of the ion channel although such an embodiment is contemplated by the present invention.

In a preferred embodiment, the ion channel is a K⁺ channel and more particularly is one or both of a G-protein-gated inward-rectifier K⁺ channel (GIRK channel) and/or an ROMK K⁺ channel.

The present invention provides, therefore, a composition such as in the form of a pharmaceutical composition useful in reducing neuronal cell death comprising an ion channel blocker such as a K⁺ channel blocker and one or more pharmaceutically acceptable carriers. Preferably, the K⁺ channel blockers inhibit the function of one or more GIRKS. Examples of such blockers include Tertiapin, Bupivicane and TEA. The composition may also comprise an agent which reduces the levels of or the activity of p75^{NTR} or a domain thereof.

The present invention further contemplates the use of an ion channel blocker such as a blocker in the manufacture of a medicament in the prevention or at least reduction of neuronal cell death or other forms of degeneration. Other molecules may also be co-administered such as a cytokine (e.g. leukemia inhibitory factor) and/or a range of genetic molecules.

The identification of a key component in p75^{NTR}-mediated or Chopper-mediated cell apoptosis permits the generation of diagnostic agents useful in assessing the functionality of K⁺ channels which may in turn determine the likelihood or otherwise of neurological damage following induction of p75^{NTR}.

The present invention further defines antagonists of molecules which promote or induce p75^{NTR}- or Chopper-mediated K⁺ channel activation. For example, K⁺ channel activators may be identified as molecules which bind to K⁺ channels such as GIRKs after activation by p75^{NTR} or Chopper. Antagonists of these molecules are proposed to be useful therapeutic agents to prevent or reduce neuronal cell death. In a further embodiment, agents are used which promote intracellular cleavage of p75^{NTR}. Intracellular forms of p75^{NTR} are proposed to mediate cell survival. Intracellular forms of p75^{NTR} may also be generated by genetic means. Such genetic means may be permanent or transient.

Alternatively, protein supplementation may be used to introduce intracellular forms of p75^{NTR}. In that case, the intracellular form of p75^{NTR} may need to be modified or co-administered with a molecule to permit entry through the membrane.

The present invention contemplates, therefore, a method for the treatment or prophylaxis of neurological damage following, for example, neurodegenerative disease or trauma. Such treatment may be the treatment of chronic conditions over a period of time or may be acute treatment such as at the site of an accident or trauma or in a triage condition.

A summary of sequence identifiers used throughout the subject specification is provided in Table 1.

TABLE 1
Summary of sequence identifiers

| SEQUENCE ID NO: | DESCRIPTION |
|---|---|
| 1 | oligonucleotide |
| 2 | oligonucleotide |
| 3 | Amino acid sequence of Tertiapin |

### BRIEF DESCRIPTION OF THE FIGURES

FIG. 1 is a diagrammatic representation providing a summary of K⁺ channels blocked by certain inhibitors.

FIG. 2 is a graphical representation showing that the Chopper domain requires external K⁺ but not Ca²⁺ to induce cell death.

FIG. 3 is a graphical representation showing the Chopper domain-mediated killing is inhibited by TEA.

FIG. 4 is a graphical representation showing the Chopper domain-mediated killing is inhibited by Bupivicane.

FIG. 5 is a graphical representation showing the Chopper domain-mediated killing is inhibited by Tertiapin.

FIG. 6 is a graphical representation showing the Chopper domain-mediated killing is inhibited by Charybdotoxin.

FIG. 7 is a graphical representation showing the Chopper domain-mediated killing is inhibited by Tertiapin.

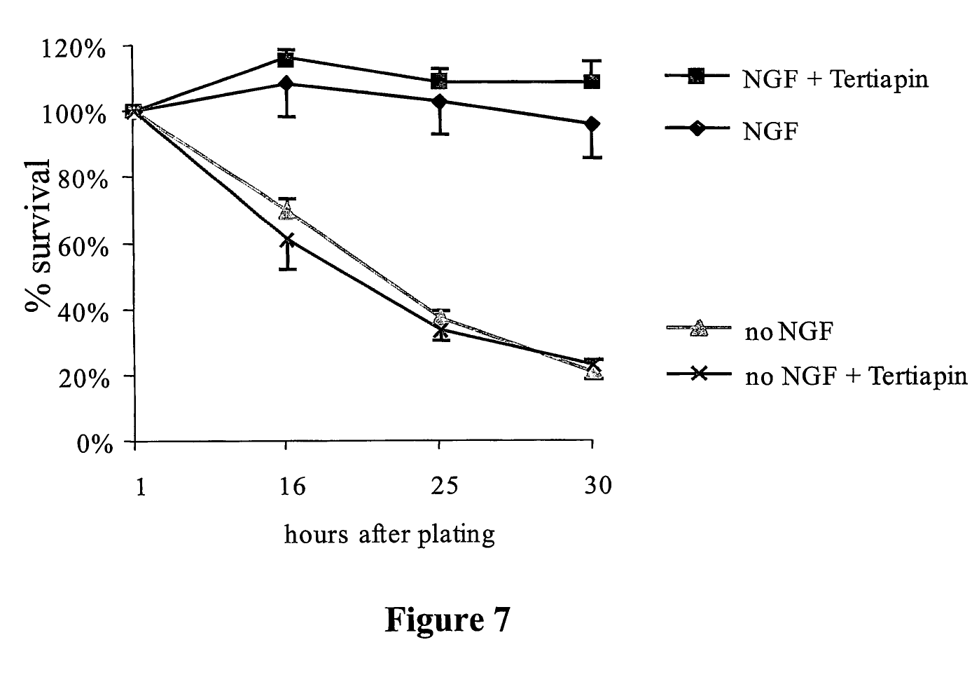

FIG. 8 is a graphical representation showing that over-expression of GIRK 2 potentiates cell death.

FIG. 9 is a graphical representation showing that Chopper mediates rubidium efflux. This indicates that Chopper-mediated death is dependent on K⁺ efflux.

FIG. 10 is a diagrammatical representation showing GIRK channels as a neuronal survival molecular switch.

FIG. 11 is a graphical representation showing that p75^{NTR}-mediated cell apoptosis requires functional GIRK channels. Apoptotic activity is measured in terms of caspase activity in the presence of Tertiapin, anti-NGF antibody and Iberiotoxin.

FIG. 12 is a graphical representation showing that p75^{NTR}-mediated cell apoptosis requires functional GIRK channels. Percentage survival of dorsal root ganglia (DRG) in the developing retina in cells with over-expression of GFP, a transmembrane plus intracellular form of p75^{NTR} (i.e. deleted extracellular domain, Δecto), Δecto and a dominant-negative (DN) GIRK and a DN GIRK. Over-expression of DN GIK inhibited Chopper-mediated apoptosis.

### DETAILED DESCRIPTION OF THE PREFERRED EMBODIMENTS

The present invention is predicated in part on the identification of a critical component of cell apoptosis, i.e. ion channelling, involved in the efflux of ions. It has been determined in accordance with the present invention that cell apoptosis and in particular neural cell apoptosis mediated via p75^{NTR} requires functional ion channels and in particular K⁺ channels. Blocking or reducing the efficacy of an ion channel has been determined, in accordance with the present invention, to prevent, inhibit or otherwise reduce cell apoptosis.

Before describing the present invention detail, it is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “an agent” includes a single agent, as well as two or more agents; reference to “an ion channel blocker” includes a single ion channel blocker as well as two or more ion channel blockers; and so forth.

In describing and claiming the present invention, the following terminology will be used in accordance with the definitions set forth below.

The terms “agent”, “compound”, “active agent”, “pharmacologically active agent”, “medicament”, “active” and “drug” are used interchangeably herein to refer to a chemical compound that induces a desired pharmacological, physiological effect. An ion channel blocker is an example of an agent, compound, active agent, pharmacologically active agent, medicament, active and drug. The terms also encompass pharmaceutically acceptable and pharmacologically active ingredients of those active agents specifically encompassed herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof.

By the terms “effective amount” or “therapeutically effective amount” of an agent as used herein are meant a sufficient amount of the agent to provide the desired therapeutic effect such as ameliorating the symptoms of neurodegenerative disease including reducing neuronal cell apoptosis. Of course, undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular neurodegenerative disorder or trauma or adverse physiological event in a susceptible individual as well as treatment of a clinically symptomatic individual by inhibiting or causing regression of a neurological disorder or disease. Thus, for example, the present method of “treating” a patient in need of therapy of the neurological system encompasses both prevention of a condition, disease or disorder as well as treating the condition, disease or disorder. In any event, the present invention contemplates the treatment or prophylaxis of any neurological condition and in particular a neurodegenerative condition. The term “condition” or “disorder” includes a disease or trauma. Treatment may be of chronic or acute conditions. Acute treatment may be at the site of an accident or trauma incident or in a triage situation such as in a location providing medical assistance.

“Patient” as used herein refers to a mammalian, preferably human, individual who can benefit from the pharmaceutical formulations and methods of the present invention. There is no limitation on the type of mammal that could benefit from the presently described pharmaceutical formulations and methods. A patient regardless of whether a human or non-human mammal may be referred to as an individual, subject, mammal, host or recipient.

Accordingly, one aspect of the present invention contemplates a method for preventing, inhibiting or otherwise reducing cell apoptosis in an animal or mammal, said method comprising administering to said animal or mammal an ion channel blocking effective amount of an agent for a time and under conditions sufficient to block or otherwise reduce the function of an ion channel.

Another aspect of the present invention contemplates a method for providing acute therapy to treat or prevent neurodegenerative damage following an accident or trauma, said method comprising administering to an animal or mammal in need of treatment an ion channel locking effective amount of an agent for a time and under conditions sufficient to block or otherwise reduce the function of an ion channel.

Reference herein to a “mammal includes a human, primate or lower primate (e.g. orangatang, marmoset), livestock animal (e.g. sheep, cow, pig, horse, donkey), laboratory test animal (e.g. mouse, rat, rabbit, guinea pig), a companion animal (e.g. dog, cat) or a captive wild animal.

The most preferred mammal is a human although the present invention particularly extends to animal models such as mouse, guinea pig, rabbit or pig models.

An “animal” includes both mammalian and non-mammalian animals. Examples of non-mammalian animals includes chickens and zebrafish.

The preferred cells in accordance with the present invention are neuronal cells or cells which express a genetic sequence encoding p75^{NTR}. Reference herein to p75^{NTR}-mediated neuronal cell apoptosis includes apoptosis induced by its Chopper domain.

Accordingly, another aspect of the present invention provides a method for preventing, inhibiting or otherwise reducing cell apoptosis in an animal or mammal wherein said cell comprises p75^{NTR}, said method comprising administering to said animal or mammal an ion channel blocking effective amount of an agent for a time and under conditions sufficient to block or otherwise reduce the function of an ion channel.

Most preferably, the cell is a neuronal cell and is capable of p75^{NTR}-mediated or Chopper-mediated apoptosis.

According to this preferred embodiment, the present invention is directed to a method for preventing, inhibiting or otherwise reducing neuronal cell apoptosis in an animal or mammal, said method comprising administering to said animal or mammal an ion channel blocking effective amount of an agent for a time and under conditions sufficient to block or otherwise reduce the function of an ion channel.

Although not wishing to limit the present invention to any one theory or mode of action, it is proposed that correct ion channel functioning is required for p75^{NTR}-mediated or Chopper-mediated neuronal cell apoptosis. One particularly important ion for p75^{NTR} mediated or Chopper-mediated apoptosis is K⁺. Consequently, p75^{NTR} mediated or Chopper-mediated neuronal cell apoptosis can be prevented, inhibited or otherwise reduced by blocking K⁺ channels.

Accordingly, another aspect of the present invention provides a method for preventing, inhibiting or otherwise reducing neuronal cell apoptosis induced or otherwise facilitated or mediated by p75^{NTR} or Chopper in an animal or mammal, said method comprising administering to said animal or mammal a K⁺ channel blocking effective amount of an agent for a time and under conditions sufficient to block or otherwise reduce the function of an K⁺ channel.

Any agent which modulates the function or activity of an ion channel and more particularly a K⁺ channel may be used in the practice of the present invention provided, of course, the agent is not overly detrimental to the overall health of the animal or mammal being treated. The agents may be chemical molecules or proteinaceous molecules such as peptides, polypeptides or proteins. Small peptides are particularly useful as are chemical analogs thereof.

Agents which inhibit G-protein-gated inward-rectifier K⁺ channels (GIRK channels) are particularly preferred as are agents which also or alternatively inhibit ROMK 2 Pore and Leak channels.

GIRK channels conduct K⁺ at or near the resting membrane potential and are involved in the control of neuron proliferation and activation. GIRK channels differ from voltage-activated K⁺ channels (Hille, B., Ionic channels of excitable membranes, Sinaur Associates, Inc. Sunderland, Mass., 1991; Ho et al., Nature 362: 127-132, 1993; Kubo et al., Nature 362: 127-132, 1993; Kubo et al., Nature 364: 802-806, 1993; Dascal et al., Proc. Natl. Acad. Sci. USA 90: 10235-10239, 1993; Krapivinsky et al., Nature 374: 135-141, 1995; Inagaki et al., Science 270: 1166-1170, 1995) and are involved in regulating resting membrane potential (Hille, 1991, supra).

Although a number of protein inhibitors of voltage-activated K⁺ channels are known (MacKinmon, R. and Miller, C, Science 245: 1382-1385, 1989; Miller, C, Neuron 1: 1003-1006, 1988; Park, C.-S. and Miller, C., Neuron 9: 307-313, 1992; MacKinnon, R. and Miller, C., Science 245: 1382-1385, 1989; MacKinnon et al., Neuron 5: 767-771, 1990; Stampe et al., Biochemistry 31: 443-450, 1994; Goldstein et al., Neuron 12: 1377-1388, 1994; Stocker, M. and Miller, C., Proc. Natl. Acad. Sci. USA 91: 9509-9513, 1994; Gross et al., Neuron 13: 961-966, 1994; Hidalgo, P. and MacKinnon, R., Science 268: 307-310, 1995; Aiyar et al., Neuron 15: 1169-1181, 1995; Rangananthan et al., Neuron 16: 131-139, 1996; Naranjo, D. and Miller, C., Neuron 16: 123-130, 1996; Gross, A. and MacKinnon, R. Neuron 16: 399-406, 1996; MacKinnon et al., Science 280: 106-109, 1998; Swartz, K. J. and MacKinnon, R., Neuron 18: 665-673, 1997; Swartz, K. J. and MacKinnon, R., Neuron 18:675-682, 1997), relatively few have been identified for the GIRK channels. One such peptide inhibitor contemplated for use in accordance with the present invention is Tertiapin (Jin W. and Lu, Z., Biochemistry 37: 13291-13299, 1998; Jin, W. and Lu, Z., Biochemistry 38: 14286-14293, 1999). Other suitable peptide inhibitors include Charybdotoxin, Bupivicane (Zhou et al., Proc. Natl. Acad. Sci. USA 98: 6482-6487, 2001) and TEA (Hille, B., Ionic channels of excitable membranes, Sinaur Associates, Lac. Sunderland, Mass., 1992, 2^{nd} edition).

Tertiapin is a small protein derived from honeybee venom and inhibits GIRK 1 and 4 subunits and ROMK1 channels with nanomolar affinities (Jin W. and Lu, Z., 1998, supra; Gauldie et al., Eur. Biochem. 61: 369-376, 1976; Ovchinnikov et al., Bioorg. Khim 6: 359-365, 1980)). The amino acid sequence of Tertiapin is ALCNCNRIIPHMCWKKCGKK (SEQ ID NO:3). A substitution of the methionine residue with a glutamine reduces oxidation of the methionine residue, rendering the molecule still stable.

Another aspect of the present invention contemplates a method for preventing, inhibiting or otherwise reducing neural cell apoptosis induced or facilitated by p75^{NTR} or a domain thereof or a homolog thereof in an animal or mammal, said method comprising administering to said animal or mammal an amount of Tertiapin or a homolog or derivative thereof effective to block or otherwise reduce the function of a GIRK channel.

The GIRK channel according to this embodiment is preferably a channel containing either GIRK 1 or 4 subunits. A particularly useful derivative of Tertiapin comprises a methionine→glutamine substitution or a functional equivalent, i.e. a mutation which reduces oxidative inactivation of the molecule. Furthermore, it is also useful to generate chemical analogs of Tertiapin as described below.

In an alternative embodiment, or it in addition to the embodiments above, the method may involve administration of other GIRK channel inhibitors such as Bupivicane, TEA and/or Charybdotoxin or their homologs, chemical analogs or derivatives. In yet another alternative, inhibitors are used to block GIRK channels directly or via intermediate or secondary components.

As stated above, chemical analogs of peptide inhibitors of K⁺ ion channels are useful due to enhanced stability and/or serum half life. Inhibitors may be proteinaceous or non-proteinaceous molecules.

Analogs of proteinaceous molecules contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinifrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-conventional amino acids

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carba- | Nnbhm |
| mylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carba- | Nnbhe |
| mylmethyl)glycine | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

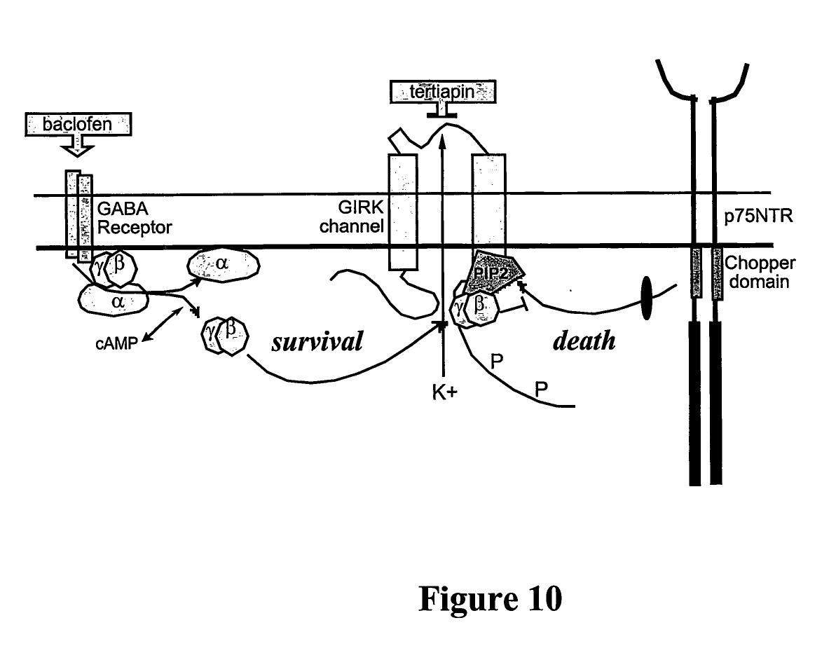

In essence, this aspect of the present invention relates to “targets” and in particular antagonists of these targets. A “target” in this instance includes a GIRK channel protein, p75^{NTR} a G-protein coupled receptor (e.g. GABA, muscarinic or opioid type receptor) compound and the like. A “Ge channel protein” includes a protein directly or indirectly associated with a GRK channel. A “GABA receptor compound” includes a component thereof such as α and β components (see FIG. 10). The term “polypeptide” refers to a polymer of amino acids and its equivalent and does not refer to a specific length of the product, thus, peptides, oligopeptides and proteins are included within the definition of a polypeptide. This term also does not refer to or exclude modifications of the polypeptide, for example, glycosylations, aceylations, phosphorylations and the like. Included within the definition are, for example, polypeptides containing one or more analogs of an amino acid (including, for example, unnatural amino acids such as defined above), polypeptides with substituted linkages as well as other modifications known in the art, both naturally and non-naturally occurring. Ordinarily, such polypeptides will be at least about 40% similar to the natural target sequence, preferably in excess of 90% and more preferably at least about 95% similar. Also included are proteins encoding by DNAs which hybridize under high or low stringency conditions to target-encoding nucleic acids and closely related polypeptides or proteins retrieved by antisera to the target protein.

Substitutional variants of target polypeptides typically contain the exchange of one amino acid for another at one or more sites within the protein and may be designed to modulate one or more properties of the polypeptide such as stability against proteolytic cleavage without the loss of other functions or properties. Amino acid substitutions may be made on the basis of similarity in polarity, charge, solubility, hydrophobicity, hydrophilicity and/or the amphipathic nature of the residues involved. Preferred substitutions are ones which are conservative, that is, one amino acid is replaced with one of similar shape and charge. Conservative substitutions are well known in the art and typically include substitutions within the following groups: glycine, alanine; valine, isoleucine, leucine; aspartic acid, glutarnic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with the target polypeptide. Since it is the interactive capacity and nature of a protein which defines that protein's biological functional activity, certain amino acid substitutions can be made in a protein sequence and its underlying DNA coding sequence and nevertheless obtain a protein with like properties. In making such changes, the hydropathic index of amino acids may be considered. The importance of the hydrophobic amino acid index in conferring interactive biological function on a protein is generally understood in the art (Kyte and Doolittle, J. Mol. Biol. 157: 105-132, 1982). Alternatively, the substitution of like amino acids can be made effectively on the basis of hydrophilicity. The importance of hydrophilicity in conferring interactive biological function of a protein is generally understood in the art (U.S. Pat. No. 4,554,101). The use of the hydrophobic index or hydrophilicity in designing polypeptides is further discussed in U.S. Pat. No. 5,691,198.

The length of the polypeptide sequences compared for homology will generally be at least about 16 amino acids, usually at least about 20 residues, more usually at least about 24 residues, typically at least about 28 residues and preferably more than about 35 residues.

The present invention provides methods of screening for drugs or other agents comprising, for example, contacting a candidate agent with a target such as a GIRK channel protein, p75^{NTR} (intracellular or extracellular portions) or a G-protein receptor component (see FIG. 10) assaying for the presence of a complex between the agent and the target. Methods well known in the art may be used. In such target binding assays, the target is typically labeled. Free target is separated from that present in a agent:target complex and the amount of free (i.e. uncomplexed) label is a measure of the binding of the agent being tested to a target. One may also measure the amount of bound, rather than free, target. It is also possible to label a ligand of the target rather than the target itself and to measure the amount of ligand binding to target in the presence and in the absence of the agent being tested.

Another technique for drug screening provides high throughput screening for compounds having suitable binding affinity to the target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with the target and washed. Bound target is then detected by methods well known in the art. This method may be adapted for screening for non-peptide, chemical entities. This aspect, therefore, extends to combinatorial approaches to screening for target antagonists.

Purified target can be coated directly onto plates for use in the aforementioned agent screening techniques. However, non-neutralizing antibodies to the target can be used to capture antibodies to immobilize the target on the solid phase.

The present invention also contemplates the use of competitive drug screening assays in which neutralizing antibodies capable of specifically binding the target compete with a test compound for binding to the target. In this manner, the antibodies can be used to detect the presence of any peptide which shares one or more antigenic determinants with the target.

The above screening methods are not limited to assays employing only target but are also applicable to studying complexes comprising targets such as membrane preparations comprising same. The effect of agents on the activity of this complex is then analyzed.

The present invention further contemplates combination therapy such as a combination of an agent to reduce the activity of p75^{NTR} and an agent to inhibit or reduce the functionality of a K⁺ channel such as GIRK.

One such agent which reduces p75^{NTR} levels is an antisense molecule or a sense molecule (or other molecule including RNAi or si-RNA) to the genetic sequence encoding p75^{NTR} or to an mRNA transcript produced by the genetic sequence. Antisense and sense suppression may also be applied to reduce proteins required for K⁺ ion channel activity.

The present invention provides, therefore, compounds such as oligonucleotides and similar species for use in modulating the function or effect of nucleic acid molecules encoding a target gene such as a gene encoding p75^{NTR} or a protein associated with GIRK channel operation (see FIG. 10), i.e. the oligonucleotides induce transcriptional or post-transcriptional gene silencing. This is accomplished by providing oligonucleotides which specifically hybridize with one or more nucleic acid molecules encoding the target or which are “sense” to the coding sequence. As used herein, the terms “target nucleic acid” and “nucleic acid molecule encoding target” have been used for convenience to encompass DNA encoding target, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

However, the present invention also contemplates sense suppression or co-suppression or RNAi-mediated or si-RNA-mediated gene silencing.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of the target gene. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of antisense technology, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid as well as “sense” equivalents for use in sense-mediated suppression. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of the invention, the present invention comprehends other families of compounds as well, including but not limited to oligonucleotide analogs and mimetics such as those described herein.

DNA and RNA molecules corresponding to the fill length of a gene may also be used.

The oligonucleotide compounds in accordance with the present invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length. However, much larger sized molecules may also be employed (e.g. full length molecules).

The open reading frame (ORF) or “coding region” which is known in the art to refer to the region between the translation initiation codon and the translation termination codon, is a region which may be targeted effectively. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′,3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2 linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Many of the preferred features described above are appropriate for sense nucleic acid molecules.

The oligonucleotides of the present invention may be administered to the target animal by any suitable means including through the intravenous, intramuscular, intranasal, rectal, intraperitoneal, intracerebral, intrathecal or subcutaneous routes; also via liposomes or retrograde transport; or locally to sites of peripheral nerve damage or injury such as using a slow release composition such as Gel-foam. As the oligonucleotides exhibit little if any toxicity to a target animal, they may be administered in any appropriate concentration provided that sufficient antisense molecules reach the target site. Appropriate ranges of concentration include, for in vivo use from about 0.01 μM to about 2,000 μM, more preferably from about 0.05 μM to about 1,500 μM and even more preferably from about 0.1 μM to about 1,000 μM. For topical use, subcutaneous use or local use, similar concentrations may be used although higher concentrations would not be deleterious to the treatment of the condition.

The oligonucleotides of the present invention may be selected for targeting almost any part of the p75^{NTR} mRNA, with the preferred oligonucleotide and length of oligonucleotide resulting in a decrease of at least about 30%, more preferably at least 50% and even more preferably at least 60% or more in the level of expression of p75^{NTR} in neurons. This also applies to other target sequences.

The preferred oligonucleotides are 5′-ACCTGCCCTCCTCATTGCA-3 (SEQ ID NO:1) which targets the 5′ end portion of the p75^{NTR} gene and 5′-AGTGGACTCGCGCATAG-3′ (SEQ ID NO:2) which targets the region comprising and/or adjacent to the termination codon of the p75^{NTR} gene including any or all mutants, derivatives, homologs or analogs thereof which are capable of hybridizing or forming a duplex with at least part of p75^{NTR} mRNA. Conveniently, the preferred oligonucleotide is a phosphorothioate oligonucleotide or is otherwise chemically modified as contemplated above.

Accordingly, another aspect of the present invention provides a composition usefull for preventing neuronal cell apoptosis, said composition comprising an oligonucleotide which is capable of:
(i) down-regulating expression of p75^{NTR} in neurons; and
(ii) hybridizing under low stringency conditions to SEQ ID NO:1 or its reverse complement; or
(iii) hybridizing under low stringency conditions to SEQ ID NO:2 or its reverse complement; and
(iv) inhibiting a K⁺ channel such as a GIRK channel, leaky channel or a ROMK channel.

This method applies to antisense and sense molecules. Another aspect of the present invention provides a method for preventing, inhibiting or otherwise reducing neural cell induced apoptosis induced or facilitated by p75^{NTR} or a domain or homolog thereof in an animal or mammal, said method comprising administering to said animal or mammal an effective amount of an agent which inhibits or otherwise impairs the functioning of a K⁺ channel and an agent which reduces the level or activity of p75^{NTR} or a domain or homolog thereof.

The agent to reduce p75^{NTR} levels and/or activity may be an agent which reduces the level of transcription or translation of the genetic sequences encoding p75^{NTR} or an agent which reduces the activity or function of the p75^{NTR} and antisense molecules, ribozymes, DNAzymes, minizyme and co-suppression or RNAi-inducing agents are particularly useful. si-RNA may also be employed.

The administration may be simultaneous or sequential. The latter includes agents being administered in either order: seconds, minutes, hours or days or weeks apart. All such agents which target the K⁺ channel or p75^{NTR} or Chopper activity or gene expression are referred to herein as “active ingredients”.

Yet another useful agent in accordance with the present invention is an antagonist of a molecule which promotes or otherwise mediates K⁺ channel activation mediated by p75^{NTR} or its domains such as Chopper. In one embodiment, p75^{NTR} activates these molecules which in turn activate a K⁺ channel such as a GIRK. The present invention, however, extends to any K⁺ ion channel, not just GIRKs. In another embodiment, p75^{NTR} activates the K⁺ channel and this leads to binding of molecules which maintain the functioning of the K⁺ channel. These can be conveniently identified by screening for the binding of molecules to K⁺ channels after p75^{NTR} or Chopper activation.

Additionally, the agent may promote generation of intracellular forms of p75^{NTR}. Such agents according to this embodiment may be agents which promote intracellular cleavage or p75^{NTR}. Alternative agents include DNA and RNA which encode intracellular forms of p75^{NTR}. Such DNA or RNA molecules may be used to promote transient or permanent expression systems for target cells. Consequently, they may comprise a promoter and means for introduction into the genome or may be expressed on a human artificial chromosome HAK).

Accordingly, the present invention provides antagonists of target molecules as well as agents which promote cleavage of intracellular p75^{NTR} and these are proposed to be useful in preventing or reducing neuronal cell death.

Accordingly, another aspect of the present invention provides a composition such as a pharmaceutical composition comprising an agent capable of inhibiting the efflux of K⁺ through a channel and optionally an agent which inhibits the function or level of p75^{NTR} or a domain or homolog thereof or which promotes formulation of intracellular p75^{NTR} and one or more pharmaceutically acceptable carriers and/or diluents.

The pharmaceutical forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of superfactants. The preventions of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization. In the case of sterile powders for the preparation of sterile injectable solutions, suitable methods of preparation include vacuum drying and the freeze-drying technique which yield a powder of active ingredient plus any additionally desired ingredient.

When the active ingredient is suitably protected, it may be orally administered, for example, with an inert diluent or with an assimilable edible carrier, or it may be enclosed in hard or soft shell gelatin capsule, or it may be compressed into tablets. For oral therapeutic administration, the active ingredient may be incorporated with excipients and used in the form of ingestible tablets, buccal tablets, troches, capsules, elixirs, suspensions, syrups, wafers and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 200 mg of active compound. Alternative dosage amounts include from about 1 μg to about 1000 mg and from about 10 μg to about 500 mg. These dosages may be per individual or per kg body weight. Administration may be per second, minute, hour, day, week, month or year.

The tablets, troches, pills and capsules and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, their use in the therapeutic compositions is contemplated. Supplementary active ingredients can also be incorporated into the compositions.

Another aspect of the present invention further contemplates the use of an inhibitor of a K⁺ channel in the manufacture of a medicament useful in the prevention or reduction in neural cell apoptosis.

The importance of the K⁺ channel in p75^{NTR}-mediated or Chopper-mediated neural cell apoptosis further enables the development of diagnostic assays. For example, an assay may be conducted to ascertain the functionality of the K⁺ channel. Subjects not having a functional K⁺ channel or where the function is impaired have a reduced risk of p75^{NTR}-mediated or Chopper-mediated neural cell apoptosis. Diagnostic assays contemplated herein include the use of labels to determine the influx or movement of K⁺ into or out of a cell as well as antibody or other K⁺ channel binding assays to determine any alteration in the conformation of the K⁺ channel.

As stated above, the preferred K⁺ channel is a GIRK channel, 2 Pore channel, a Leaky channel and/or a ROMK 1 channel. Examples of a GIRK channel is a GIRK 1, 2, 3 or 4 channel.

Natural product or chemical library screenings may also be used to identify K⁺ channel binding agents useful as either therapeutic agents and as diagnostic assay agents.

The present invention provides, therefore, a means for treating neurodegenerative disorders, diseases or conditions as well as trauma.

The present invention, therefore, contemplates a method of treating conditions such as cerebral palsy, trauma induced paralysis, vasuclar ischaemia associated with stroke, neuronal tumors, motomeurone disease, Parkinson's disease, Huntington's disease, Alzheimer's disease, multiple sclerosis and peripheral neuropathies associated with diabetes, heavy metal or alcohol toxicity, renal failure and/or infectious diseases such as herpes, rubella, measles, chicken pox, HIV and/or HTLV-1.

Whilst the present invention is particularly predicated on blocking K⁺ channel activity, another aspect of the present invention contemplates activating the K⁺ channel to a level which blocks the death signal mediated via p75^{NTR}. According to this embodiment, it is proposed that synaptic activity mimics thereof (e.g. G-protein receptor agonists such as Baclofen, cAMP or G-proteins themselves) which stimulates GIRK channel opening. Without intending to limit the present invention to any one theory or mode of action, it is proposed that GIRKs might act as a molecular switch distinguishing between survival signal activity and death signaling. Consequently, elevated activity of a GIRK may inhibit the death signal.

In addition, there may be cancers or tumors of the neurological system in which cell death is desired. Consequently, agonists of Chopper-mediated cell death mechanisms are also contemplated by the present invention.

Another aspect of the present invention provides a genetically modified animal wherein said animal produces altered levels of proteins associated with K⁺ ion channels such as but not limited to GIRKs.

The animal models of the present invention may be in the form of the animals including fish or may be, for example, in the form of embryos for transplantation. The embryos are preferably maintained in a frozen state and may optionally be sold with instructions for use.

The genetically modified animals may produce larger or smaller amounts of a target relative to non-genetically modified animals. Such animals are particularly useful animal models for screening for agents which are capable of ameliorating the physiological effects of enhanced or reduced K⁺ channelling.

A genetically modified animal includes a transgenic animal, or a “knock-out” or “knock-in” animal as well as a conditional deletion mutant. Furthermore, co-suppression may be used to induce post-transcriptional gene silencing. Co-suppression includes induction of RNAi.

The present invention is further described by the following non-limiting Examples.

### EXAMPLE 1

### Cell Culture

Dorsal Root ganglia were dissected from postnatal day zero C57B1/6 mice and plated at low density as previously described (Coulson et al., J. Biol. Chem. 274: 163787-163791, 1990) in Nunc terasaki plates. Cells were grown in MonomedII media (CSL, Melbourne) with 1% v/v serum in nerve growth factor (2.5 S NGF 50 ng/ml) and allowed to adhere overnight before experimentation. Alternative media included DMEM and DMEM calcium free media (Sigma), PBS (ingredients), and HBBS (ingredients). In order to prevent indiscritinant cell death over the experimental period 0.1% v/v serum and 50 ng/ml NGF was included in all solutions.

### EXAMPLE 2

### Inhibitors

BAPTA and BAPTA-AM were purchased from CalBiochem. Tertiapin, δ-denrotoxin, rCharybdotoxin, rIberotoxin, rMargatoxin and Nifedipine were purchased from Alomone Laboratories. Tetraethylammonium chloride (TEA), 4-aminopyridine (4-AP) and Pertussis toxin were purchased from Sigma. Inhibitors were dissolved in either di-methyl sulphoxide. DMSO or pure water according to manufacturer's recommendations. Cultures were never exposed to greater than 0.1% w/v DMSO.

### EXAMPLE 3

### Peptide and Inhibitor Treatments

The number of live neurons was counted prior to the addition of Penetratin linked peptides (t-0). Cells were incubated with 1-3 μM of inhibiting peptide for two hours then the cells were counted within half an hour of removal of peptides, three washes and replacement of media. Where experiments were performed in media other than Monomed, cells were washed at least three times in the new media before addition of fresh media containing the peptide/inhibitor. Inhibitors were added to the cells just prior to the Chopper peptide addition or were pretreated for 5-30 minutes. Cells were pretreated with Pertussis toxin 18 hours prior to Chopper peptide addition. Neuronal viability was determined by phase bright, robust morphology and propidium iodide exclusion.

### EXAMPLE 4

### Peptides

Peptides were synthesized, conjugated to fluorescein, palmitoyl, and Penetratin (Derossi et al., Trends Cell Biol. 8: 84-87, 1998) and purified as previously described (Coulson et al., 2000, supra). Where a Chopper-derived peptide did not contain a cysteine within the sequence capable of participating in di-sulfide bond formation with Penetratin, an hydroxy-terminal cys was added to the peptide prior to addition of the lys-palmitoyl and fluorescein. Both an unrelated palnitoylated control peptide palmgp130pen (Coulson et al., 2000, supra) and the inactive Chopper peptides (PalmC) were used as non-death inducing control peptides: palngp130pen was used in experiments addressing the role of calcium, palnCpen was used in cell culture experiments using channel inhibitors.

### EXAMPLE 5

### Inhibitors of K⁺ Channels

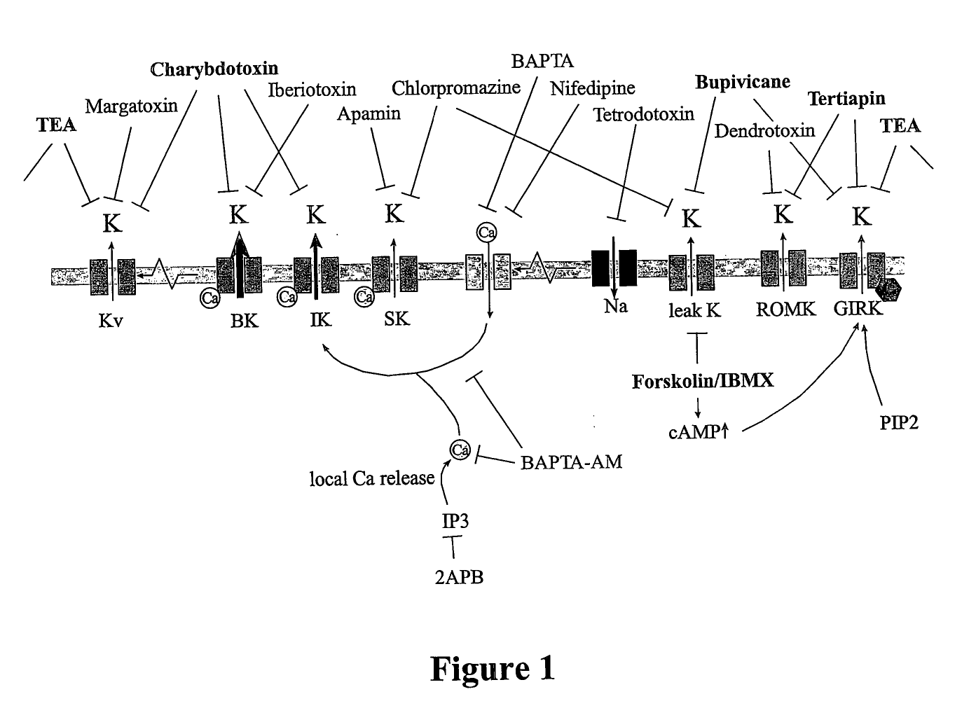

FIG. 1 provides a summary of K⁺ channel inhibitors. The K⁺ channels inhibited by Tertiapin, Bupivicane and Charybdotoxin are shown.

### EXAMPLE 6

### Comparison of Channel Inhibitors on p75^{NGRF}-Mediated Apoptosis of Neuronal Cells

Table 3 provides a list of ion channel inhibitors and whether or not they inhibit p75^{NR}-(i.e. Chopper)-mediated apoptosis.

TABLE 3

| Inhibitor | Activity | Prevention of killing? |
|---|---|---|
| BAPTA-AM | Intracellular Ca⁺chelator | Yes |
| BAPTA | Extracellular Ca⁺chelator | No |
| Nefidipine | Voltage gated Ca⁺ | No |
| 2-APB | IP3 mediated Ca⁺release | No |
| Iberiotoxin | Large Ca⁺K⁺(IK) | No |
| Apamin | Small Ca⁺K⁺ | No |
| Charybdotoxin | Kv+; Large Ca⁺K+ | Yes |
| TEA | K⁺channels | Yes |
| 4-aminopyridine | Kv channels | Toxic |
| Margatoxin | Kv1.3 | No |
| IBMX/Forskolin | K⁺leak channels, cAMP | Yes |
| TTX | Na⁺channels | No |
| TEA | Kv and GIRK channels | Yes |
| Bupivicane | GIRKs K+ leak channels | Yes |
| Tertiapin | GIRK 1, 2 & 4, ROMK | Yes |
| δ-Dendrotoxin | ROMK | No |
| Pertusis toxin | G proteins | No |

### EXAMPLE 7

### Requirement of K⁺ for p75^{NTR}-Mediated Cell Death

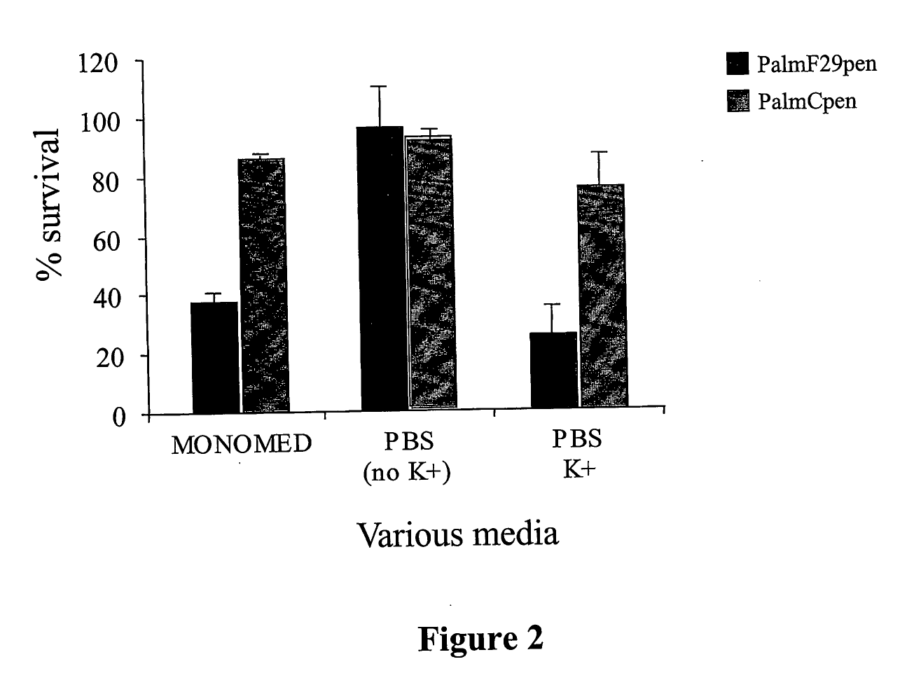

FIG. 2 shows that p75^{NTR} requires external K⁺ to mediate cell apoptosis.

### EXAMPLE 8

### p75^{NTR}-Mediated Apoptosis of Cells is Inhibited by TEA

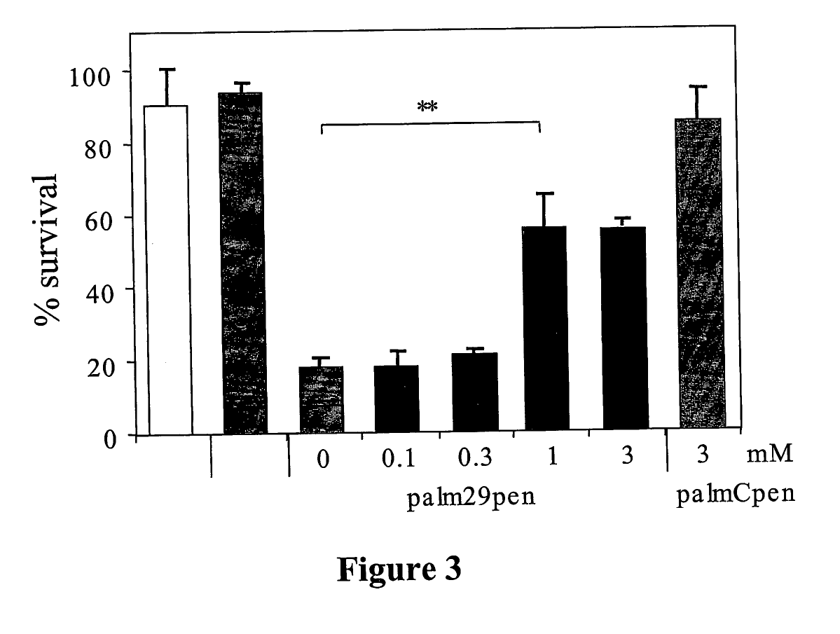

FIG. 3 shows that p75^{NTR} (i.e. Chopper)-mediated cell apoptosis is inhibited by TEA.

### EXAMPLE 9

### p75^{NTR}-Mediated Apoptosis of Cells is Inhibited by Bupivicane

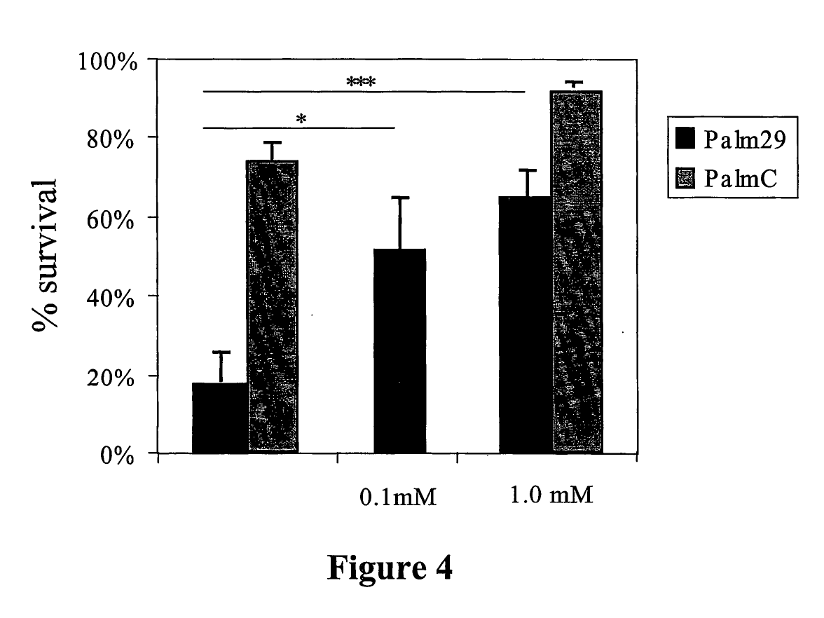

FIG. 4 shows that p75^{NTR} (i.e. Chopper)-mediated cell apoptosis is inhibited by Bupivicane.

### EXAMPLE 10

### p75^{NTR}-Mediated Apoptosis of Cells is Inhibited by Tertiapin

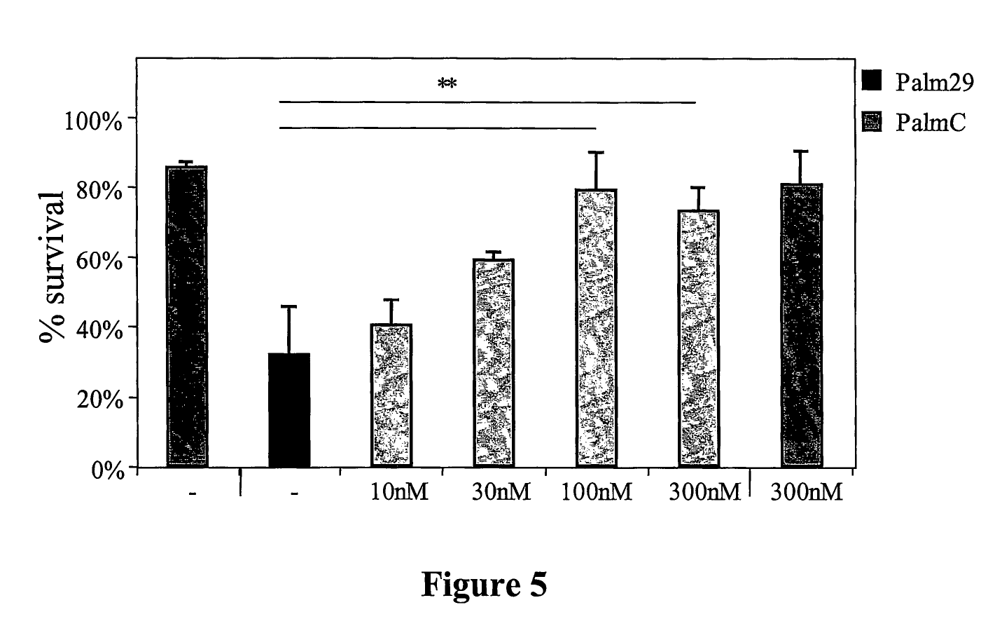

FIG. 5 shows that p75^{NTR} (i.e. Chopper)-mediated cell apoptosis is inhibited by Tertiapin.

### EXAMPLE 11

### p75^{NTR}-Mediated Apoptosis of Cells is Inhibited by Charybdotoxin

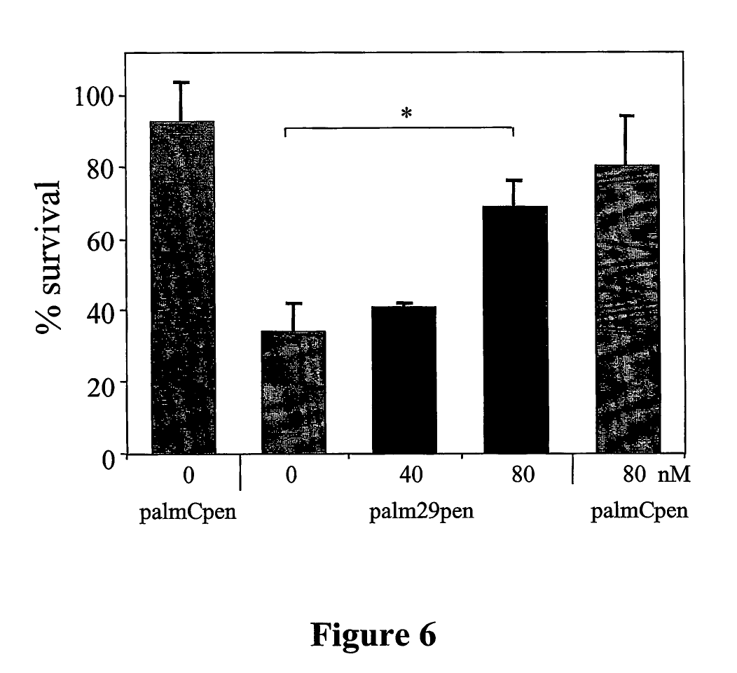

FIG. 6 shows that p75^{NTR} (i.e. Chopper)-mediated cell apoptosis is inhibited by Charybdotoxin.

### EXAMPLE 12

### Over-Expression of GIRK 2 in Cells

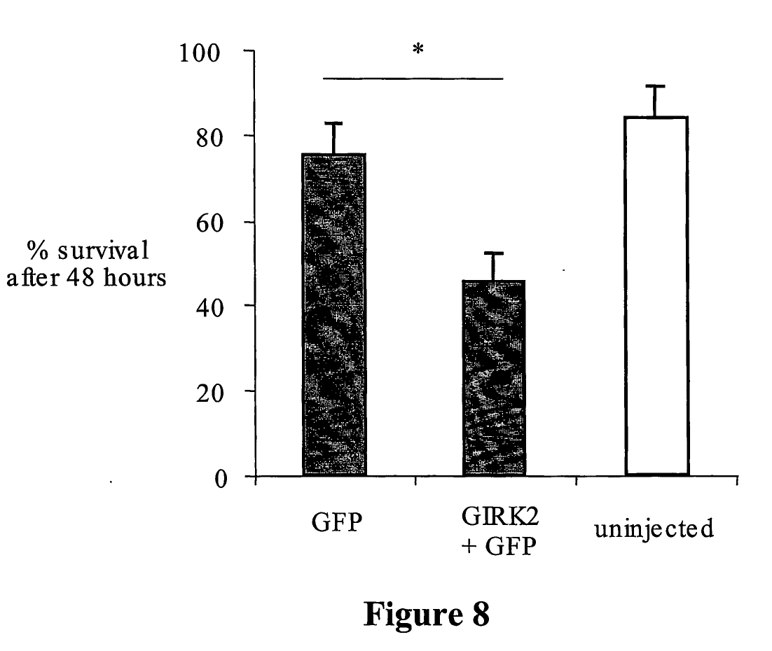

FIG. 8 shows that over-expression of GIRK 2 potentiates cell death.

### EXAMPLE 13

### Activation of K⁺ Channel Activation by p75^{NTR} or Chopper

A mixture of biological molecules are unabated with p75NTR or Chopper and the ability for any of these molecules to bind to a GIRK is assessed. These molecules are proposed to be activated by p75^{NTR} or Chopper and in turn activate a GIRK. It is further proposed that antagonists of these molecules prevent or reduce neuron cell death.

### EXAMPLE 14

### Chopper Mediates K⁺ Efflux

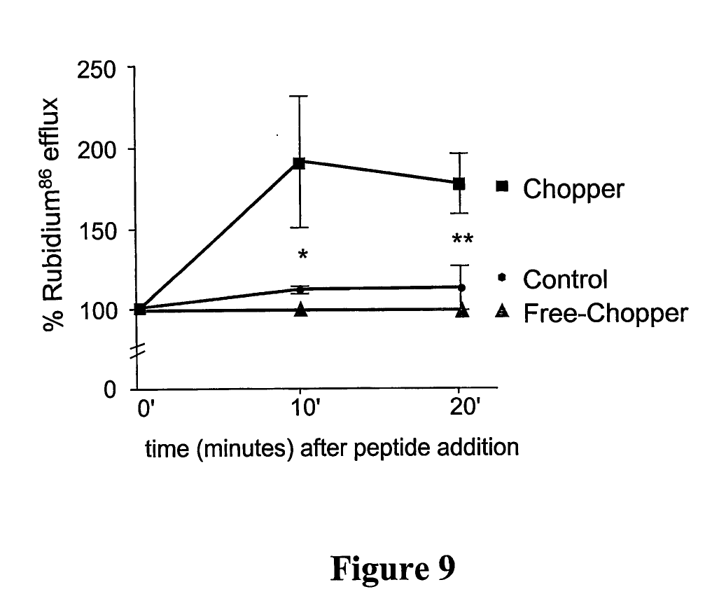

FIG. 9 is a graphical representation showing that Chopper causes rubidium efflux and, therefore, K⁺ efflux. Consequently, Chopper-mediated cell death is dependent on K⁺ efflux.

### EXAMPLE 15

### Model of GIRK Channels as a Neuronal Survival Molecular Switch

FIG. 10 provides a model of GIRK channels as a neuronal survival molecular switch. Blocking of the βγ subunit by pertussis toxin still results in Chopper-mediated killing. However, GABA receptor agonists such as Baclofen which have the effects of activating GIRKs result in promotion of cell survival.

### EXAMPLE 16

### Intracellular p75^{NTR} Promotes Cell Survival

Intracellular cleavage of p75^{NTR} or introduction of DNA or RNA which encodes intracellular p75^{NTR} promotes cell survival. In one embodiment, DNA capable of expression via its own promoter or using cell transcription/translation machinery encoding intracellular p75^{NTR} is used to generate transient peptide, sufficient to promote cell survival.

### EXAMPLE 17

### p75^{NTR}-Mediated Apoptosis Requires Functional GIRK Channels

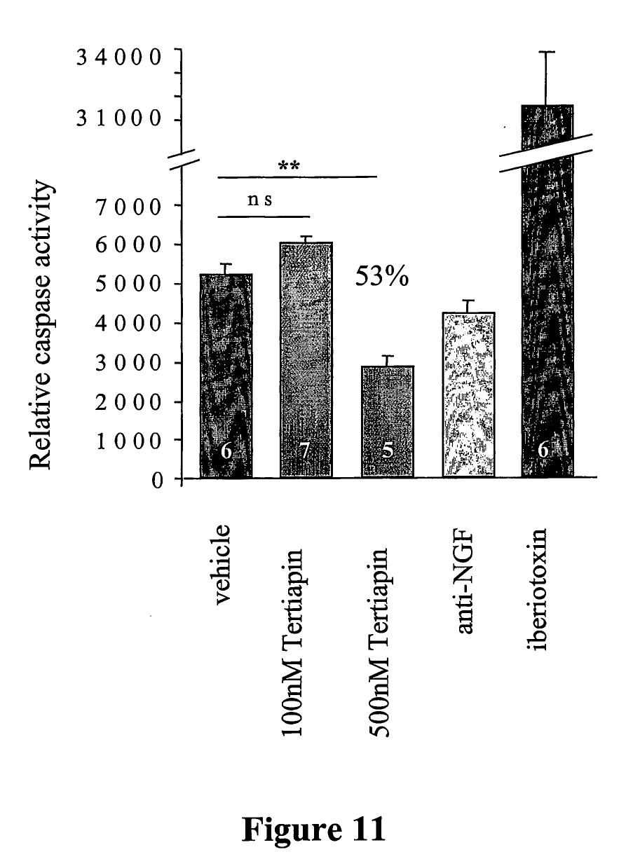

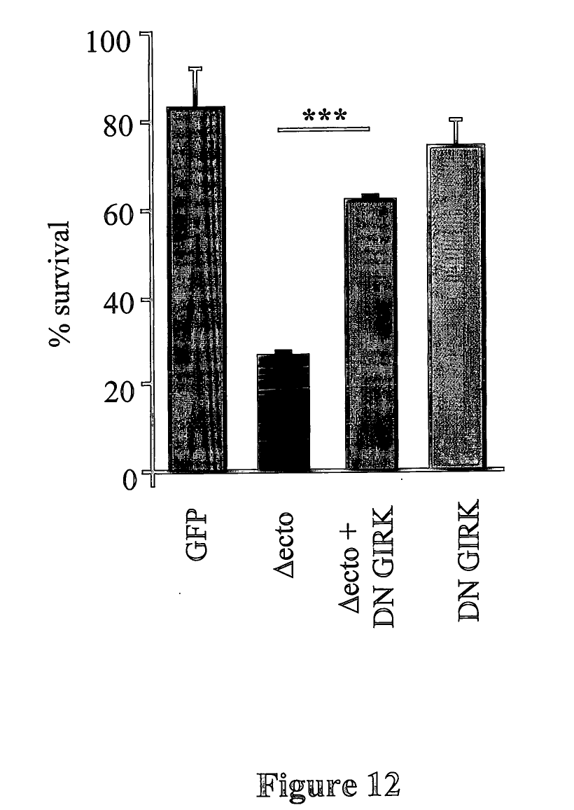

FIGS. 11 and 12 show results indicating that p75^{NTR}-mediated apoptosis requires functional GIRK channels. In FIG. 11, Tertiapin was injected into the eye of chick embryos. Anti-NGF antibodies and Iberiotoxin (a Ca⁺⁺ channel inhibitor) were also tested. Apoptotic activity was measured using caspase activity. 500 nM of Tertiapin and anti-NGF antibodies were effective in reducing caspase activity. As Tertiapin is a GERK channel blocker, this indicates that functional GIRK channels are required for p75^{NTR}-mediated cell apoptosis.

FIG. 12 shows the effects of dorsal root ganglia (DRG) in which dominant-negative (GN) GIRKs are over-expressed alone or with GFP, Δecto (p75^{NTR} less its extracellular domain) or Δecto alone. Again, over-expression of the GIRK overcame p75^{NTR} cell death.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Aiyar et al., Neuron 15: 1169-1181, 1995
Banks et al., J. Comp. Neurol. 429: 156-165, 2001
Bortner et al., J. Biol. Chem. 276: 4304-4314, 2001
Coulson et al., J. Biol. Chem. 274: 163787-163791, 1990
Coulson et al., J. Biol. Chem. 275: 30537, 30545, 2000
Dallaporta et al., J. Immunol. 160: 5605-5615, 1998
Dascal et al., Proc. Natl. Acad. Sci. USA 90: 10235-10239, 1993
Derossi et al., Trends Cell Biol. 8: 84-87, 1998
Gauldie et al., Eur. Biochem. 61: 369-376, 1976
Goldstein et al, Neuron 12: 1377-1388, 1994
Gross and MacKinnon, Neuron 16.: 399-406, 1996
Gross et al., Neuron 13: 961-966, 1994
Hidalgo and MacKinnon, Science 268: 307-310, 1995
Hille, B., Ionic channels of excitable membranes, Sinaur Associates, Inc. Sunderland, Mass., 1991
Hille, Ionic channels of excitable membranes, Sinaur Associates, Inc. Sunderland, Mass., 1992, 2^{nd} edition)
Ho et al., Nature 362: 127-132, 1993
Hughes et al., J. Biol. Chem. 272: 30567-30576, 1997
Inagaki et al., Science 270: 1166-1170, 1995
Jin and Lu, Biochemistry 377: 13291-13299, 1998
Jin and Lu, Biochemistry 38: 14286-14293, 1999
Krapivinsky et al., Nature 374: 135-141, 1995
Kubo et al., Nature 362: 127-132, 1993
Kubo et al., Nature 364: 802-806, 1993
Kyte and Doolittle, J. Mol. Biol. 157: 105-132, 1982
MacKinnon and Miller, Science 245: 1382-1385, 1989
MacKinnon and Miller, Science 245: 1382-1385, 1989
MacKinnon et al., Neuron 5: 767-771, 1990
MacKinnon et al., Science 280: 106-109, 1998
Miller, Neuron 1: 1003-1006, 1988
Naranjo and Miller, Neuron 16.: 123-130, 1996
Ovchinnikov et al., Bioorg. Khim 6: 359-365, 1980
Park and Miller, Neuron 9: 307-313, 1992
Pereira et al., Int. J. Dv. Neurosci. 19: 559-567, 2001
Rangananthan et al., Neuron 16: 131-139, 1996
Stampe et al., Biochemistry 31: 443-450, 1994
Stocker and Miller, Proc. Natl. Acad. Sci. USA 91: 9509-9513, 1994
Swartz and MacKinnon, Neuron 18: 665-673, 1997
Swartz and MacKinnon, Neuron 18:675-682, 1997
Terrado et al., J. Neurosci. 21: 3144-3510, 2001
Wang et al., J. Biol. Chem. 274: 3678-3685, 1999
Yu and Choi, Proc. Natl. Acad. Sci. USA 97: 9360-9362, 2000
Zhou et al., Proc. Natl. Acad. Sci. USA 98: 6482-6487, 2001

1. A method for preventing, inhibiting or otherwise reducing cell apoptosis in an animal or mammal, said method comprising administering to said animal or mammal an ion channel blocking effective amount of an agent for a time and under conditions sufficient to block or otherwise reduce the function of an ion channel.

2. The method of claim 1 wherein the mammal is a human, livestock animal, companion animal, laboratory test animal or a captured wild animal.

3. The method of claim 2 wherein the mammal is a human.

4. The method of claim 1 or 2 or 3 wherein the cells are neuronal cells.

5. The method of claim 4 wherein the method prevents, inhibits or otherwise reduces p75^{NTR} or Chopper-mediated neuronal cell apoptosis.

6. The method of any one of claims 1 to 5 wherein the ion channel is a channel.

7. The method of claim 6 wherein the K⁺ channel is a G-protein-gated inward-rectifier K⁺ channel (GIRK channel).

8. The method of claim 6 wherein the K⁺ channel is a leak channel.

9. The method of claim 6 wherein the K⁺ channel is ROMK.

10. The method of claim 1 wherein the agent is Tertiapin or a homolog or derivative thereof.

11. The method of claim 1 wherein the agent is TEA or a homolog or derivative thereof.

12. The method of claim 1 wherein the agent is Bupivicane or a homolog or derivative thereof.

13. The method of claim 1 wherein the agent is Charybdotoxin or a homolog or derivative thereof.

14. The method of any one of claims 1 to 13 further comprising the administration of an agent which down-regulates the level or activity of p75^{NTR} or a domain or homolog thereof.

15. The method of claim 14 wherein the second agent is a genetic molecule.

16. The method of claim 15 wherein the genetic molecule is an antisense or sense molecule to the genetic sequence encoding p75^{NTR} or its Chopper domain.

17. A method for preventing, inhibiting or otherwise reducing cell apoptosis in an animal or mammal, said method comprising administering to said animal or mammal an effective amount of an agent which promotes cleavage of an intracellular portion of p75^{NTR} or which otherwise promotes generation of a free intracellular form of p75^{NTR}.

18. The method of claim 17 wherein the agent is a intracellular cleavage agent.

19. The method of claim 17 wherein the agent is RNA or DNA encoding an intracellular portion of p75^{NTR}.

20. The method of claim 17 or 18 or 19 wherein the mammal is a human, livestock animal, companion animal, laboratory test animal or a captured wild animal.

21. The method of claim 20 wherein the mammal is a human.

22. An isolated antagonist of a molecule which, following activation by p75^{NTR} or a domain thereof is capable of activating a K⁺ channel.

23. The antagonist of claim 22 wherein the K⁺ channel is a GIRK.

24. The antagonist of claim 22 wherein the domain of p75^{NTR} is Chopper.

25. A composition such as a pharmaceutical composition comprising an agent capable of inhibiting the efflux of K⁺ through a channel and optionally an agent which inhibits the function or level of p75^{NTR} or a domain or homolog thereof and one or more pharmaceutically acceptable carriers and/or diluents.

26. A composition comprising the antagonist of any one of claims 22 to 24 and one or more pharmaceutically acceptable carriers and/or diluents.

27. Use of Tertiapin, TEA, Bupivicane, Charybdotoxin or other antagonist of any one of claims 22 to 24 or homologs or analogs thereof in the manufacture of a medicament for the treatment or prophylaxis of a neurodegenerative disease or trauma.

28. A method for providing acute therapy to treat or prevent neurodegenerative damage following an accident or trauma, said method comprising administering to an animal or mammal in need of treatment an ion channel blocking effective amount of an agent for a time and under conditions sufficient to block or otherwise reduce the function of an ion channel.

29. The method of claim 28 wherein the mammal is a human, livestock animal, companion animal, laboratory test animal or a captured wild animal.

30. The method of claim 29 wherein the mammal is a human.

31. The method of claim 28 or 29 or 30 wherein the cells are neuronal cells.

32. The method of claim 31 wherein the method prevents, inhibits or otherwise reduces p75^{NTR}- or Chopper-mediated neuronal cell apoptosis.

33. The method of any one of claims 29 to 32 wherein the ion channel is a channel.

34. The method of claim 33 wherein the K⁺ channel is a G-protein-gated inward-rectifier K⁺ channel (GIRK channel).

35. The method of claim 33 wherein the K⁺ channel is a leak channel.

36. The method of claim 33 wherein the K⁺ channel is ROMK.

37. The method of claim 28 wherein the agent is Tertiapin or a homolog or derivative thereof.

38. The method of claim 28 wherein the agent is TEA or a homolog or derivative thereof.

39. The method of claim 28 wherein the agent is Bupivicane or a homolog or derivative thereof.

40. The method of claim 28 wherein the agent is Charybdotoxin or a homolog or derivative thereof.

41. The method of any one of claims 28 to 40 further comprising the administration of an agent which down-regulates the level or activity of p75^{NTR} or a domain or homolog thereof.

42. The method of claim 41 wherein the second agent is a genetic molecule.

43. The method of claim 42 wherein the genetic molecule is an antisense or sense molecule to the genetic sequence encoding p75^{NTR} or its Chopper domain.

44. The method of claim 28 further comprising the co-administration of a cytokine.

45. The method of claim 44 wherein the cytokine is leukemia inhibitory factor.
